(19) United States
(12) Patent Application Publication
(10) Pub. No.: US 2024/0175834 A1
(43) Pub. Date: May 30, 2024

(54) Method for Measuring Alkalinity of Water Sample

(71) Applicant: Foshan Viomi Electrical Technology Co., Ltd., Foshan (CN)

(72) Inventors: Su Lv, Foshan (CN); Hongchen Dong, Foshan (CN); Ru Chen, Foshan (CN); Bo Yan, Foshan (CN)

(21) Appl. No.: 18/430,595

(22) Filed: Feb. 1, 2024

Related U.S. Application Data
(63) Continuation of application No. PCT/CN22/70087, filed on Jan. 4, 2022.

(30) Foreign Application Priority Data
Aug. 3, 2021 (CN) 202110885140.1

(51) Int. Cl. G01N 27/06; G01N 33/18

(52) U.S. Cl. CPC G01N 27/06; G01N 33/18

(57) ABSTRACT

Provided in the present disclosure is a method for measuring alkalinity of water sample, including following steps: step 1: constructing a mathematical model correlating the alkalinity of a water body with a water body electrical signal variation value; step 2: bringing a water sample into contact with an acidic material to cause a change in a water sample electrical signal value, collecting the water sample electrical signal value before and after the water sample is brought into contact with the acidic material, and constructing a water sample electrical signal variation value; and step 3: deriving the alkalinity of the water sample by substituting the water sample electrical signal variation value into the water body electrical signal variation value in the mathematical model. The above method does not involve using titrant and prior calibration operation, which does not require high-level hardware equipment.

### CROSS-REFERENCE TO RELATED APPLICATIONS

The present application is a continuation application of PCT application No. PCT/CN2022/070087 filed on Jan. 4, 2022, which claims priority of Chinese Patent Application No. 202110885140.1 filed on Aug. 3, 2021 before CNIPA. All the above are hereby incorporated by reference in their entirety as part of the present disclosure.

### FIELD

The present disclosure relates to the technical field of water quality testing and, particularly, a method for measuring alkalinity of water sample.

### BACKGROUND

Water is the origin of life. Human beings cannot live and work without water, and the quality of drinking water is closely related to human health. With social and economic development, scientific progress, and the improvement of people's living standards, people's requirements for the quality of drinking water are increasingly improved, and the standard of drinking water quality is also correspondingly and continuously developed and perfected. Water alkalinity is a comprehensive characteristic indicator of water, which is an important indicator for judging water quality and water processing control. Alkalinity is also commonly used for evaluating the buffering capacity of water bodies and the solubility and toxicity of metals therein.

Existing water alkalinity testing methods include the following: acid-base titration, potentiometric titration, and spectrophotometric methods. These methods are cumbersome and complex to operate and generally involve the use of a chemical titrant to assist the measurement, a calibration step prior to the formal measurement, and constant maintenance of the instrument. However, the accuracy of the above test methods is still difficult to control. Individual operational differences of the testers, contamination or aging of the equipment may cause the test results to suffer from deviations that are not negligible. Based on the various problems in the existing water alkalinity testing methods, the popularization and application of water alkalinity testing technology in life and production is advanced.

### SUMMARY

In accordance with an aspect of the present disclosure, provided is a method for measuring alkalinity of water sample, including following steps: step 1: constructing a mathematical model correlating the alkalinity of a water body with a water body electrical signal variation value, the water body electrical signal variation value comprising at least one of an electrical conductivity variation value or a function constructed by an electrical conductivity variation value; step 2: bringing a water sample into contact with an acidic material to cause a change in a water sample electrical signal value, collecting the water sample electrical signal value before and after the water sample is brought into contact with the acidic material, and constructing a water sample electrical signal variation value; and step 3: deriving the alkalinity of the water sample by substituting the water sample electrical signal variation value into the water body electrical signal variation value in the mathematical model. Alkaline ions such as carbonate ions, bicarbonate ions, and hydroxide ions in the water body contribute to the alkalinity of the water body, and in the present disclosure, the alkalinity of the water body or the alkalinity of the water sample referred to is a total amount of alkalinity in water capable of depleting the acidic material. Based on the depletion of alkalinity-contributing ions in the water body, it leads to a decrease in the ionic concentration of the water body and a consequent change in electrical conductivity. An acidic material is introduced into the present disclosure so that alkalinity-contributing ions in the water sample are depleted by neutralization reactions with the acidic material to produce a change in electrical signal value of the water sample. The change in electrical signal value based on the neutralization reaction directly reflects the content of alkalinity-contributing ions, and the alkalinity value of water samples is obtained quickly and accurately by constructing a derived relationship between water alkalinity and the electrical signal of the water body. The above method does not involve using titrant and prior calibration operation, which does not require high-level hardware equipment. Therefore, it saves the cumbersome and complicated routine maintenance of the instrument, and is superior in simple operation, high reliability, and environmental protection. Water body electrical signals include the electrical performance parameters of the water body, such as conductivity, resistivity, voltage, current, potential and other parameters that directly characterize the electrical signals of the water body, as well as the parameters and functions that are derived and constructed based on the electrical performance parameters of the water body. It should be noted that the acidic materials used in the present disclosure are materials that are not easily soluble in water. The water body electrical signal variation value is constructed in the above method as a variable for deriving the alkalinity of the water body, and the above variable is in a strongly correlated linear relationship with the alkalinity of the water body, in which the linear coefficient of the constructed linear relationship is up to 0.99 or more, so that the alkalinity of the water body is accurately derived by utilizing the linear relationship obtained by fitting the above variable to the alkalinity of the water body.

Based on the alkalinity contributing ions being conductive, proposed in the present disclosure is a method for measuring alkalinity of water sample by deriving the alkalinity of water samples from water sample electrical signal variations. Therefore, in the reaction of depleting alkalinity-contributing ions, the selection of the ionic species participating in the reaction, the selection of the parameters of the electrical signals, and the correction of the error factors all have a positive effect on the accuracy of the alkalinity values of the water samples derived by the above method.

### BRIEF DESCRIPTION OF THE DRAWINGS

FIG. 1 shows a fitting plot of a linear relationship regarding the correlation between the conductivity variation value and the alkalinity value of water samples constructed by adopting a weak acid hydrogen type resin in Example 2;

FIG. 2 shows a fitting plot of a linear relationship regarding the correlation between the TDS variation value and the alkalinity value of water samples constructed by adopting a weak acid hydrogen type resin in Example 3.

### DETAILED DESCRIPTION OF THE EXAMPLES

For a better understanding of the solutions of the present disclosure by those skilled in the art, the technical solutions in the examples of the present disclosure are clearly and completely described and discussed below. Obviously, the examples described herein are only some of the examples of the present disclosure but not all of them.

The objective of the present disclosure is to provide a method for measuring alkalinity of water sample, so that water alkalinity is measured accurately in a simple operation.

Preferably, a variable of the function includes a total amount of dissolved solid. Preferably, the dissociation constant pKa of the acidic material satisfies pKa>2.

Preferably, the dissociation constant pKa of the acidic material satisfies pKa>3.

Preferably, the dissociation constant pKa of the acidic material satisfies pKa>4.

Adopting the dissociation constant as one of the considered indicators for the selection of acidic materials is conducive to ensuring the sensitivity of the reaction between the acidic materials and the water body, thereby improving the measurement efficiency of the alkalinity of the water body.

Preferably, the acidic material satisfies that a change in electrical signal contributed by the acidic material leaching substances in a test solution is less than 50% of a change in electrical signal due to alkalinity. The dissolution of acidic materials in water may affect the conductivity of water, which may bring errors to the alkalinity measurement of water based on the electrical signal variation. When selecting acidic materials, the ability of acidic materials to contribute to the conductivity of water is taken as one of the indicators to be considered, which is conducive to the improvement of the accuracy of alkalinity measurement of water.

Preferably, the acidic material includes acidic resins, the acidic resins being selected from at least one of sulfonic acid resins, carboxylic acid resins, phosphoric acid resins, boronic acid resins, and silicic acid resins.

Preferably, the acidic resins include the carboxylic acid resins.

Preferably, the acidic material includes a non-ionic material and an anionic substance, the non-ionic material is used to provide an acidic non-ionic group, the non-ionic group is denoted by A-H, the anionic substance is denoted by A-, and in terms of a molar ratio, an amount ratio of A- to A-H ranges from 0 to 99%.

Preferably, the content of R-A-H in acidic resins ranges from 0.01 mmol/g to 50 mmol/g.

Preferably, the content of R-A-H in acidic resins ranges from 0.5 mmol/g to 5 mmol/g. Preferably, the content of R-A-H in acidic resins ranges from 2 mmol/g to 4 mmol/g.

Preferably, in terms of molar ratio, an amount ratio of A- to A-H ranges from 0.001 to 1%.

An anionic resin is compounded with a non-ionic resin, thereby reducing the conductivity contribution of the acidic resin to the water body, which is conducive to improving the accuracy of the alkalinity measurement of the water body.

Preferably, the acidic material contains 50 ppm-50,000 ppm of metal ions. Optionally, the metal ions are selected from at least one of alkali metal ions, alkaline-earth metal ions, and transition metal ions. Bringing the metal ion content in the acidic material within the above range ensures that the alkalinity measurement offers a quick response.

Preferably, a reaction volume ratio of the water sample to the acidic material ranges from 0.1 to 10 in the step 2. Within the above ratio, the water sample and the acidic material may fully interact with each other in a short period, so that the conductivity of the water sample may change significantly, thereby ensuring that the alkalinity measurement offers a quicker response.

Preferably, the step 2 further includes: measuring a water temperature on the water sample, correcting a measured electrical signal by temperature based on the water temperature of the water sample to obtain a corrected electrical signal, and calculating the water sample electrical signal variation value by substituting the measured electrical signal with the corrected electrical signal; a rule for temperature correction is that for each increase of the water temperature by 1° C. compared to 25° C., a value of 1 to 5% increase of the electrical signal is used as the corrected electrical signal, and for each decrease of the water temperature by 1° C., a value of 1 to 5% decrease of the electrical signal is used as the corrected electrical signal value.

Preferably, the electrical signal is the electrical conductivity. A rule for temperature correction is that for each increase of the water temperature by 1° C. compared to 25° C., a value of 2% increase of the electrical signal is used as the corrected electrical signal, and for each decrease of the water temperature by 1° C., a value of 2% decrease of the electrical signal is used as the corrected electrical signal value.

The gradient alkalinity value water samples adopted in the following examples are taken from tap water across the country, the alkalinity of the water body is measured by the Guangzhou Institute of Microbiology as full alkalinity (i.e., methyl orange alkalinity), the conductivity variation value is measured by the conductivity meter of the U.S. Mellon Company, and the TDS value is measured by the TDS pen of Xiaomi.

### Example 1

### (1) Verification of the Contribution of Different Types of Resins to the Conductivity of Water Samples

The conductivity variation of different types of resins after soaking pure and tap water samples for 24 hours is applied, and the experimental results data are shown in Table 1. The data provided in Table 1 show that the strong acid hydrogen type resin (sulfonic acid resin) exchanges with cations in water to generate strong acids, resulting in a sharp increase in solution conductivity; the weak acid potassium type resin (carboxylic acid resin) dissolves more ions when soaked in pure water. However, the strong acid sodium type resin (sodium sulfonate resin) and the weak acid hydrogen type (carboxylic acid resin) do not cause significant fluctuations in conductivity when soaked in pure water samples, which indicates that the strong acid sodium type resin and the weak acid hydrogen type resin contribute little to the conductivity variation of the water samples. Further, the above four participating resins are soaked in tap water and changes in conductivity of the water samples are observed. The data in Table 1 show that neither strong acid sodium type resin nor weak acid potassium type resin causes significant changes in the conductivity of tap water when soaked in tap water, respectively. In contrast, the strong acid hydrogen type resin and the weak acid hydrogen type resin cause significant changes in the conductivity of tap water after they are soaked in the tap water. In summary, among the four resins provided in the present example, only the weak acid hydrogen type resin simultaneously satisfies the following: it hardly contributes to the conductivity of the pure water sample (the conductivity variation value of pure water after being soaked in the weak acid hydrogen type resin is almost zero), and it reacts sufficiently with the tap water sample to cause a significant change in the conductivity of the tap water sample. Based on the above, weak acid hydrogen type resins are suitable for application in water quality monitoring based on conductivity variation. The weak acid resin cleaned with pure water is basically free from the influence of dissolved substances, its conductivity is the most sensitive to the response of water samples, and it is equally effective even for low TDS water samples.

TABLE 1
Contribution of different types of resins to
the conductivity variation of water samples

| | Conductivity | Conductivity |
|---|---|---|
| | variation | variation |
| | after soaking | after soaking |
| Resin types | in pure water | in tap water |
| Strong acid hydrogen type | 230 uS | >200% |
| Strong acid sodium type | <10 uS | 1.0% |
| Weak acid hydrogen type | <10 uS | 43.1% |
| Weak acid potassium type | 110 uS | 0.6% |

(2) Verification that Weak Acid Hydrogen Type Resins Only Change Conductivity in Response to Water Sample Alkalinity

Based on the above experimental results, the weak acid hydrogen type resin is more advantageous than the other participating resins in the application of water quality monitoring based on conductivity variation. The weak acid hydrogen type resin is mixed overnight with sodium chloride or calcium chloride solution configured with pure water (alkalinity is only 20 ppm), the water samples are measured for hardness, alkalinity, and conductivity before and after soaking the weak acid hydrogen type resin, and the relevant data are recorded in Table 2. It is found that the hardness of the water samples does not change. However, the alkalinity of the water samples changes significantly, thereby indicating that the weak acid hydrogen resin reacts only with the substances contributing to alkalinity in the water samples and does not exchange or attract other cations, resulting in an accurate reflection of the alkalinity value of the water samples.

TABLE 2
Results of solution immersion experiments
for weak acid hydrogen type resins

| | Water | Alka- | conduc- |
|---|---|---|---|
| Composition of solutions | Hardness | linity | tivity |
| 600 ppm NaCl solution (control) | 0 | 20 | 1192 |
| 600 ppm NaCl solution 200 g + weak acid | 0 | 0 | 1430 |
| hydrogen resin 15 g (soaked overnight) | | | |
| 600 ppm CaCl₂ solution (control) | 290 | 22 | 1160 |
| 600 ppm CaCl₂ solution 200 g + weak acid | 290 | 0 | 1516 |
| hydrogen resin 15 g (soaked overnight) | | | |

### Example 2

The mathematical model applied to the method for measuring alkalinity of water samples involved in the present example is constructed as follows:

S1. Water samples with gradient alkalinity values (as shown in Table 3) are used as gradient standards, and the conductivity of each gradient standard is measured respectively;

S2. Each gradient standard is mixed with the acidic material respectively and stirred together for about 1 h;

S3. The conductivity of each gradient standard is measured;

S4. As for the same standard, the conductivity measured for the first time is taken as C1, the conductivity measured for the second time is taken as C2, and the conductivity variation values (i.e., C2−C1) before and after the reaction of each gradient standard with the acidic material are organized, so that a linear relationship curve is fitted with the conductivity variation values as the horizontal coordinates and the alkalinity values of the water samples as the vertical coordinates. The linear relationship curve obtained is used as a mathematical model to correlate the alkalinity and conductivity variation values of the water body.

TABLE 3
Gradient standards and their corresponding alkalinity values

| Source of Reference Standards | Alkalinity (mg/L) |
|---|---|
| Tap water at site A | 95.1 |
| Tap water at site B | 102.0 |
| Tap water at site C | 35.0 |
| Tap water at site D | 25.0 |
| Tap water at site E | 112.0 |
| Tap water at site F | 49.0 |
| Tap water at site G | 110.0 |
| Tap water at site H | 270.0 |
| Tap water at site I | 114.0 |
| Tap water at site J | 260.0 |
| Tap water at site K | 118.0 |

Different types of acidic resins are used as acidic materials for constructing a mathematical model in accordance with the present example documented herein to construct a mathematical model for correlating the conductivity variation of water samples and the alkalinity value of water samples. The acidic resins used in the present example are commercially available acidic resins, and the participating resins are as follows: weak acid hydrogen type resin I (Purolite C107E, carboxylate resin), weak acid hydrogen type resin II (Dupont Amberlite IRP-64, carboxylate resin), and weak acid sodium type resin (Ningbo Zhengguang ZGC152, carboxylate resin). The acidic resin involved is subjected to the following pre-treatment before use: weighing a certain mass of the acidic resin and soaking it in deionized water in order to remove the dissolved matter in the acidic resin, continuously soaking it with constant stirring, and fishing out the acidic resin and washing the acidic resin with a large amount of deionized water and fishing it out for spare use after about 12 h. In the above pre-treatment step, reducing the conductivity contribution of the dissolved material to the water body is conducive to improving the accuracy of the alkalinity measurement of the water body.

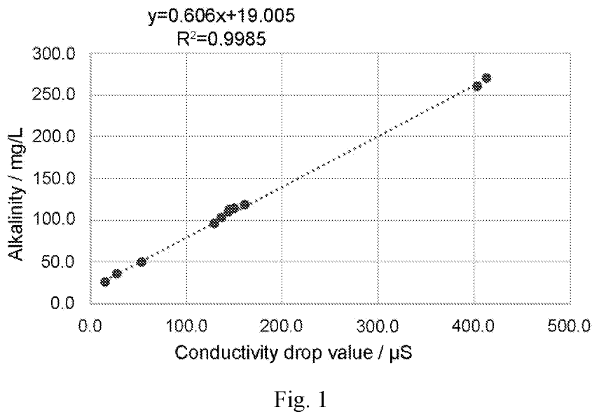

In the above pre-treatment step, the reduction of the conductivity contribution of the dissolved material to the water body is conducive to improving the accuracy of the alkalinity measurement of the water body. The linear relationship fitted therefrom is shown in FIG. 1, which achieves an R2 of 0.9985, indicating that the alkalinity of water samples shows a strong linear relationship with the conductivity variation value of water samples.

TABLE 4
Data involved in the construction of linear relationships
using weak acid hydrogen type resins

| | Gradient standards | | | | |
|---|---|---|---|---|---|
| | | | | Theoret- | |
| | | Raw | Conduc- | ical | |
| | | water | tivity | conduc- | |
| | Alka- | conduc- | drop | tivity | Devi- |
| | linity | tivity | value | drop value | ation/ |
| | (mg/L) | (uS) | (uS) | (uS) | % |
| Tap water at site A | 95.1 | 293.0 | 129.4 | 124.3 | 4.0% |
| Tap water at site B | 102.0 | 277.0 | 136.8 | 135.0 | 1.3% |
| Tap water at site C | 35.0 | 89.7 | 28.4 | 30.7 | −8.2% |
| Tap water at site D | 25.0 | 90.0 | 15.7 | 15.1 | 3.6% |
| Tap water at site E | 112.0 | 388.0 | 145.8 | 150.6 | −3.3% |
| Tap water at site F | 49.0 | 156.0 | 54.6 | 52.5 | 3.9% |
| Tap water at site G | 110.0 | 367.5 | 144.4 | 147.5 | −2.1% |
| Tap water at site H | 270.0 | 850.0 | 414.0 | 396.6 | 4.2% |
| Tap water at site I | 114.0 | 373.0 | 150.0 | 153.7 | −2.5% |
| Tap water at site J | 260.0 | 1003.0 | 404.0 | 381.0 | 5.7% |
| Tap water at site K | 118.0 | 413.0 | 161.0 | 159.9 | 0.7% |

Water samples are tested for alkalinity using the weak acid hydrogen type resin of the present example and the linear relationship shown in FIG. 1, as follows:

S1. Before testing the water samples, the conductivity of the test water is measured with a conductivity meter and recorded as C1, and the acidic material is mixed with the test water and stirred together for about 1 h. The mass of the test water is 80 g, the dried acidic material is 15 g, and the conductivity of the water is measured after stirring for 1 h and recorded as C2.

S2. The conductivity variation value (i.e., C2−C1) is calculated before and after the reaction of the water sample with the acidic material, and the conductivity variation value calculated therefrom is applied to the linear relationship shown in FIG. 1 to calculate the alkalinity value of the water sample.

The results of the above calculations are shown in Table 5, and none of the resulting deviations of the calculated values of alkalinity from the measured values of alkalinity exceed 10% in absolute value.

TABLE 5
Derivation of alkalinity values of water samples
applying the linear relationship shown in FIG. 1

| | | Alkalinity | Alkalinity | |
|---|---|---|---|---|
| | Conductivity | calculated | measured | |
| | drop value | value | value | Deviation/ |
| Sample Number | (uS) | (mg/L) | (mg/L) | % |
| Water Sample a | 145.8 | 112 | 107.3 | 4.2% |
| Water Sample b | 54.6 | 49 | 51.9 | 6.1% |
| Water Sample c | 404 | 260 | 264.0 | 1.6% |
| Water Sample d | 414 | 270 | 270.1 | 0.0% |
| Water Sample e | 136.8 | 102 | 101.9 | 0.1% |

### Example 3

In the present example, a weak acid sodium type resin and a weak acid hydrogen type resin are mixed in different ratios and then soaked in tap water, and the TDS value of the water samples is measured when the soaking time reaches 60 min. The weak acid hydrogen type resins and weak acid sodium type resins involved are subjected to the following pre-treatment before use: weighing a certain mass of the weak acid hydrogen type resins and weak acid sodium type resins and soaking them in deionized water in order to remove the dissolved matter in the resin, continuously soaking them with constant stirring, and fishing out the acidic resin and washing the acidic resin with a large amount of deionized water and fishing them out for spare use after about 12 h. In the above pre-treatment step, reducing the conductivity contribution of the dissolved material to the water body is conducive to improving the accuracy of the alkalinity measurement of the water body. Before soaking the resin, the TDS value of the raw water sample used in the present example is 140 ppm. As shown in Table 6, after soaking in tap water for 60 min, there is no change in the conductivity of the water sample corresponding to the 100% sodium resin, while the TDS corresponding to the mixed resin consisting of the sodium and hydrogen resins decreases to 81-88 ppm due to the removal of alkalinity.

TABLE 6
Contribution of the ratio of weak acid sodium resin and weak
acid hydrogen resin to the water sample TDS variation

| weak acid sodium type resin: weak acid | TDS value (ppm) after 60 |
|---|---|
| hydrogen type resin (molar ratio/%) | min soaking in tap water |
| raw water sample | 140 |
| 100% | 140 |
| 59.6% | 88 |
| 37.0% | 84 |
| 22.9% | 82 |
| 2.9% | 81 |
| 0% | 82 |

### Example 4

Based on the experimental results of Example 2 and Example 3, a weak acid hydrogen type resin I and a weak acid sodium type resin (Ningbo Zhengguang ZGC152, a carboxylic acid type resin) are used to compound a mixed resin (10:1 in weight ratio) to react with the water sample, resulting in the conductivity variation value of the water sample showing a strong linear relationship with the alkalinity of the water sample. The weak acid hydrogen type resin used in Example 2 is used in the present example, and a mathematical model for correlating a TDS variation of water samples and an alkalinity value of water samples is constructed with reference to the method for constructing a mathematical model for alkalinity measurement of water samples provided in Example 2. The specific operations are as follows:

S1. Water samples with gradient alkalinity values (as shown in Table 3) are used as gradient standards, and the conductivity of each gradient standard is measured respectively;

S2. Each gradient standard is then mixed together with the compounded mixing resin respectively and stirred for about 1 h;

S3. The TDS value of each gradient standard is then measured;

S4. As for the same standard, the TDS value measured for the first time is taken as T1, the TDS value measured for the second time is taken as T2, and the TDS variation values (i.e., T2−T1) before and after the reaction of each gradient standard with the compounded mixing resin are organized, so that a linear relationship curve is fitted with the TDS variation values as the horizontal coordinates and the alkalinity values of the water samples as the vertical coordinates. The linear relationship curve obtained is used as a mathematical model to correlate the alkalinity and TDS variation values of the water body.

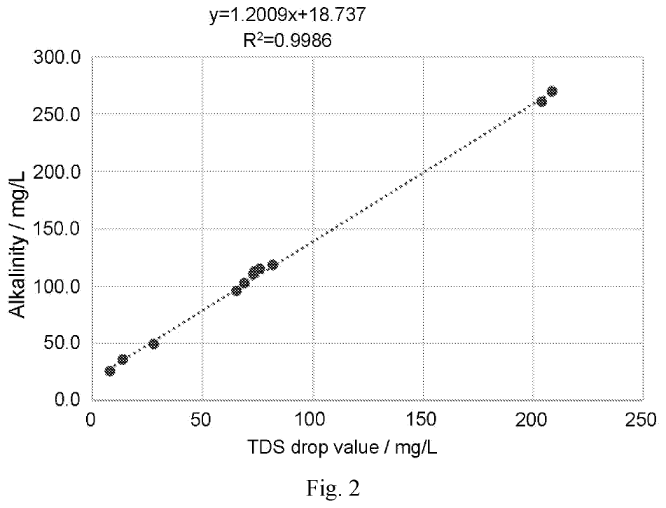

The data involved in the construction of the above linear model are shown in Table 7. The linear relationship fitted therefrom is shown in FIG. 2, which achieves an R2 of 0.9986, indicating that the alkalinity of water samples shows a strong linear relationship with the TDS variation value of water samples.

TABLE 7
Relationship between TDS drop and alkalinity of water samples

| | Alkalinity | TDS drop value | Raw water TDS |
|---|---|---|---|
| Sample Number | (mg/L) | (mg/L) | (mg/L) |
| Tap water at site A | 95.1 | 66 | 148 |
| Tap water at site B | 102.0 | 69 | 140 |
| Tap water at site C | 35.0 | 14 | 46 |
| Tap water at site D | 25.0 | 8 | 46 |
| Tap water at site E | 112.0 | 74 | 195 |
| Tap water at site F | 49.0 | 28 | 79 |
| Tap water at site G | 110.0 | 73 | 185 |
| Tap water at site H | 270.0 | 209 | 429 |
| Tap water at site I | 114.0 | 76 | 188 |
| Tap water at site J | 260.0 | 204 | 504 |
| Tap water at site K | 118.0 | 82 | 208 |

Alkalinity measurement of water samples is performed using the weak acid hydrogen type and weak acid sodium type mixed resins in the present example and the linear relationship shown in FIG. 2, as follows:

S1. Before testing the water samples, the TDS value of the test water is measured with a TDS pen and recorded as T1, and the acidic material is mixed with the test water and stirred together for about 1 h. The mass of the test water is 80 g, the dried acidic material is 15 g, and the TDS value of the water is measured after stirring for 1 h and recorded as T2.

S2. The TDS variation value (i.e., T2−T1) is calculated before and after the reaction of the water sample with the acidic material, and the TDS variation value calculated therefrom is applied to the linear relationship shown in FIG. 2 to calculate the alkalinity value of the water sample.

The results of the above calculations are shown in Table 8, and none of the resulting deviations of the calculated values of alkalinity from the measured values of alkalinity exceed 5% in absolute value.

TABLE 8
Derivation of alkalinity values of water samples
applying the linear relationship shown in FIG. 2

| | | Alkalinity | Alkalinity | |
|---|---|---|---|---|
| | TDS | calculated | measured | |
| | drop value | value | value | Deviation/ |
| Sample Number | (mg/L) | (mg/L) | (mg/L) | % |
| Water Sample a | 66 | 97.4 | 95.1 | 2.4% |
| Water Sample b | 14 | 35.8 | 35.0 | 2.4% |
| Water Sample c | 73 | 106.4 | 110.0 | −3.3% |
| Water Sample d | 209 | 269.7 | 270.0 | −0.1% |
| Water Sample e | 82 | 117.2 | 118.0 | −0.7% |

The above examples are only used to illustrate the technical solution of the present disclosure rather than to limit the protection scope of the present disclosure. Although the present disclosure has been described in detail with reference to the above examples, a person of ordinary skill in the art should be understood that modifications or equivalent substitutions may be carried out to the technical solution of the present disclosure without departing from the essence and scope of the technical solutions of the present disclosure.

1. A method for measuring alkalinity of water sample, comprising following steps:
step 1: constructing a mathematical model correlating the alkalinity of a water body with a water body electrical signal variation value, the water body electrical signal variation value comprising at least one of an electrical conductivity variation value or a function constructed by an electrical conductivity variation value;
step 2: bringing a water sample into contact with an acidic material to cause a change in a water sample electrical signal value, collecting the water sample electrical signal value before and after the water sample is brought into contact with the acidic material, and constructing a water sample electrical signal variation value; and
step 3: deriving the alkalinity of the water sample by substituting the water sample electrical signal variation value into the water body electrical signal variation value in the mathematical model.

2. The method for measuring alkalinity of water sample according to claim 1, wherein a variable of the function comprises a total amount of dissolved solid.

3. The method for measuring alkalinity of water sample according to claim 1, wherein a dissociation constant pKa of the acidic material satisfies pKa>2.

4. The method for measuring alkalinity of water sample according to claim 3, wherein the dissociation constant pKa of the acidic material satisfies pKa>4.

5. The method for measuring alkalinity of water sample according to claim 4, wherein the acidic material satisfies that a change in electrical signal contributed by acidic material leaching substances in a test solution is less than 50% of a change in electrical signal due to alkalinity.

6. The method for measuring alkalinity of water sample according to claim 5, wherein the acidic material comprises acidic resins, the acidic resins being selected from at least one of sulfonic acid resins, carboxylic acid resins, phosphoric acid resins, boronic acid resins, and silicic acid resins.

7. The method for measuring alkalinity of water sample according to claim 6, wherein the acidic resins comprise the carboxylic acid resins.

8. The method for measuring alkalinity of water sample according to claim 6, wherein the acidic material comprises a non-ionic material and an anionic substance, the non-ionic material is used to provide an acidic non-ionic group, the non-ionic group is denoted by A-H, the anionic substance is denoted by A⁻, and in terms of a molar ratio, an amount ratio of A⁻ to A-H ranges from 0 to 99%.

9. The method for measuring alkalinity of water sample according to claim 6, wherein the acidic material contains 50 ppm-50,000 ppm of metal ions.

10. The method for measuring alkalinity of water sample according to claim 9, wherein a reaction volume ratio of the water sample to the acidic material ranges from 0.1 to 10 in the step 2.

11. The method for measuring alkalinity of water sample according to claim 1, wherein the step 2 further comprises:
measuring a water temperature on the water sample, correcting a measured electrical signal by temperature based on the water temperature of the water sample to obtain a corrected electrical signal, and calculating the water sample electrical signal variation value by substituting the measured electrical signal with the corrected electrical signal; and
a rule for temperature correction is that for each increase of the water temperature by 1° C. compared to 25° C., a value of 1 to 5% increase of an electrical signal is used as the corrected electrical signal, and for each decrease of the water temperature by 1° C., a value of 1 to 5% decrease of the electrical signal is used as the corrected electrical signal value.
